(19) United States
(12) Patent Application Publication
(10) Pub. No.: US 2019/0289957 A1
(43) Pub. Date: Sep. 26, 2019

(54) TOE SEPARATING DEVICE AND FOOTWEAR HAVING THE SAME

(71) Applicant: Chih-Ling LEE, New Taipei City (TW)

(72) Inventor: Chih-Ling LEE, New Taipei City (TW)

(21) Appl. No.: 16/355,896

(22) Filed: Mar. 18, 2019

(30) Foreign Application Priority Data
Mar. 22, 2018 (TW) 107109775

(51) Int. Cl. A43B 7/26; A61F 5/01

(52) U.S. Cl. CPC A43B 7/26; A61F 5/019

(57) ABSTRACT

A footwear includes a sole and a toe separating device disposed on a top surface of the sole. The toe separating device includes a hallux separating unit having a hallux separating column for separating the hallux and the second toe of a user's foot. The hallux separating column has a hallux columnar body defining a receiving space with an opening formed in a top surface thereof. The hallux columnar body has a length in a top-bottom direction and a width in a left-right direction transverse to the top-bottom direction. A hallux adjusting member is removably inserted into the receiving space through the opening to expand the width of the hallux columnar body.

### CROSS-REFERENCE TO RELATED APPLICATION

This application claims priority of Taiwanese Invention Patent Application No. 107109775, filed on Mar. 22, 2018.

### FIELD

The disclosure relates to a toe separating device and a footwear having the same.

### BACKGROUND

Inappropriate size of footwear and incorrect walking posture are among the factors causing toe bone deformities, such as hallux valgus and overlapping toes, which are usually accompanied by skewed body posture, resulting in soreness in other parts of the body. In order to separate the toes, a patient usually wears a toe separating device.

Referring to FIG. 1, a conventional toe separating device 1 as disclosed in Taiwanese Patent No. M532853, includes a main body 11, a hallux separating portion 12 and three toe separating portions 13 disposed on the main body 11, an arcuate upper connecting portion 14 interconnecting top surfaces of the hallux separating portion 12 and the toe separating portions 13, and an arcuate lower connecting portion 15 interconnecting bottom surfaces of the hallux separating portion 12 and the toe separating portions 13. By virtue of the hallux separating portion 12 and the toe separating portions 13, the toes of a user are spread to thereby achieve a relaxing effect of the foot.

However, because the hallux separating portion 12 of the conventional toe separating device 1 has a fixed shape and hardness, it cannot be changed or adjusted to match severe toe bone deformities of the patient. This causes excessive pain and discomfort during use of the conventional toe separating device 1, such that the conventional toe separating device 1 cannot be worn by the user for a long period of time. Furthermore, patients having mild symptoms of toe bone deformities also gain no benefit by wearing the conventional toe separating device 1.

### SUMMARY

Therefore, an object of the disclosure is to provide a toe separating device and a footwear having the same that can alleviate at least one of the drawbacks of the prior art.

According to an aspect of the disclosure, a toe separating device includes a hallux separating unit including a hallux separating column for separating the hallux and the second toe of a user's foot, and a hallux adjusting member. The hallux separating column has a hallux columnar body that is elastically stretchable and that defines a first receiving space having an opening formed in a top surface of the hallux columnar body. The hallux columnar body has a length in a top-bottom direction, and a width in a left-right direction transverse to the top-bottom direction. The hallux adjusting member has a columnar shape and is removably inserted into the first receiving space through the opening to expand the width of the hallux columnar body.

According to another aspect of the disclosure, a footwear includes a sole, and a toe separating device disposed on a top surface of the sole and including a hallux separating unit. The hallux separating unit includes a hallux separating column for separating the hallux and the second toe of a user's foot, and a hallux adjusting member. The hallux separating column has a hallux columnar body that is elastically stretchable and that defines a first receiving space having an opening formed in a top surface of the hallux columnar body. The hallux columnar body has a length in a top-bottom direction, and a width in a left-right direction transverse to the top-bottom direction. The hallux adjusting member has a columnar shape and is removably inserted into the first receiving space through the opening to expand the width of the hallux columnar body.

### BRIEF DESCRIPTION OF THE DRAWINGS

Other features and advantages of the present disclosure will become apparent in the following detailed description of the embodiments with reference to the accompanying drawings, of which:

FIG. 1 is a perspective view of a conventional toe separating device disclosed in Taiwanese Patent No. M532853;

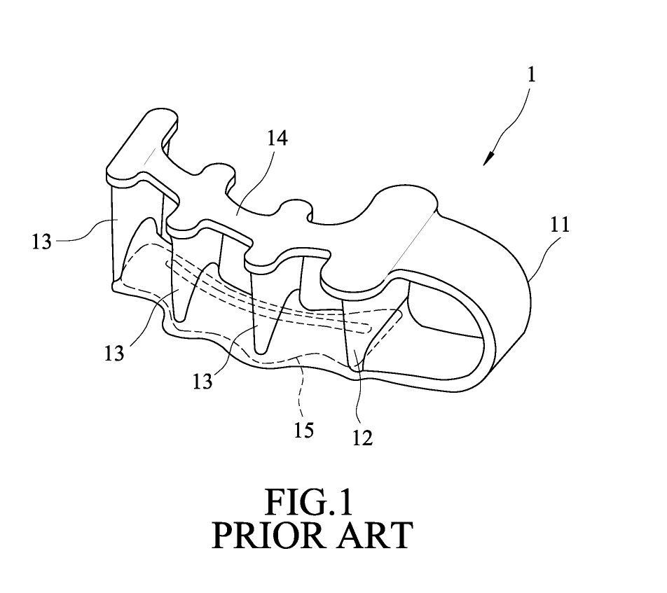

FIG. 2 is a perspective view of a first embodiment of a footwear having a toe separating device according to the present disclosure;

FIG. 3 is a perspective view of the toe separating device of the first embodiment;

FIG. 4 is an exploded sectional view of the toe separating device of the first embodiment;

FIG. 5 is a view similar to FIG. 4, but illustrating the toe separating device in an assembled state; and

FIG. 6 is an exploded perspective view of a second embodiment of a footwear having a toe separating device according to the present disclosure.

### DETAILED DESCRIPTION

Before the present disclosure is described in greater detail, it should be noted that where considered appropriate, reference numerals or terminal portions of reference numerals have been repeated among the figures to indicate corresponding or analogous elements, which may optionally have similar characteristics.

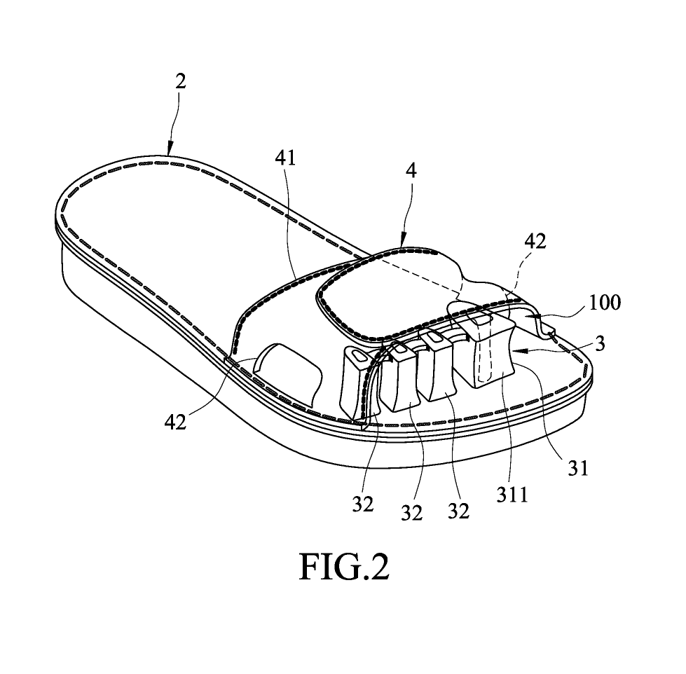

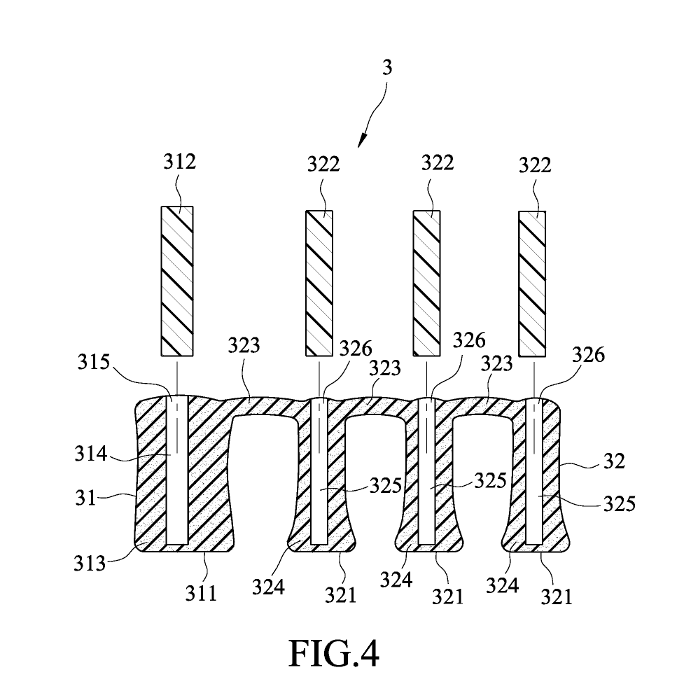

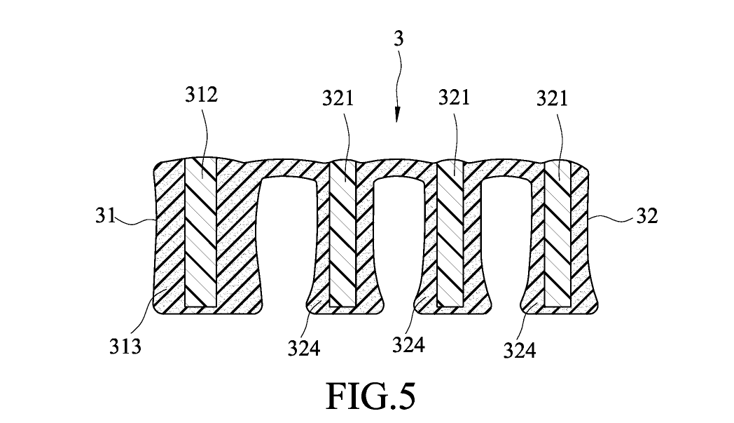

Referring to FIGS. 2 to 5, a first embodiment of a footwear according to the present disclosure includes a sole 2, a toe separating device 3, and an upper 4. In this embodiment, the footwear is exemplified as a slipper.

The toe separating device 3 is disposed on a top surface of the sole 2, and includes a hallux separating unit 31, three toe separating units 32, and three arched connecting members 323. The hallux separating unit 31 includes a hallux separating column 311, and a hallux adjusting member 312. The hallux separating column 311 is used for separating the hallux and the second toe of a user's foot, and has a hallux columnar body 313 that is elastically stretchable and that defines a first receiving space 314 having an opening 315 formed in a top surface thereof. The hallux columnar body 313 has a length in a top-bottom direction, and a width in a left-right direction transverse to the top-bottom direction.

The hallux adjusting member 312 has a columnar shape and is removably inserted into the first receiving space 314 through the opening 315 to expand the width of the hallux columnar body 313.

Each toe separating unit 32 includes a toe separating column 321 and a toe adjusting member 322. The toe separating column 321 is used for separating two adjacent toes excluding the hallux, and has a toe columnar body 324 that is elastically stretchable and that defines a second receiving space 325 having an opening 326 formed in a top surface thereof. The toe columnar body 324 has a length in the top-bottom direction and a width in the left-right direction.

The toe adjusting member 322 has a columnar shape and is removably inserted into the second receiving space 325 through the opening 326 of the second receiving space 325 to expand the width of the toe columnar body 324. Each of the toe separating column 321 and the toe adjusting member 322 has a diameter between 2 mm and 12 mm.

One of the arched connecting members 323 interconnects the toe separating column 321 of one of the toe separating units 32 and the hallux separating column 311. Each of the other two of the arched connecting members 323 interconnects the toe separating columns 321 of two adjacent ones of the toe separating units 32. The hallux separating column 311 and the toe separating columns 321 are arranged spaced apart from each other along the left-right direction.

It should be noted herein that each of the hallux columnar body 313 and the toe columnar body 324 is made of a soft and flexible material, such as foam, rubber, silicone, etc. Through this, the hallux columnar body 313 can fittingly receive the hallux adjusting member 312 made of a rigid material and having different widths to thereby expand the width of the hallux columnar body 311, and each toe columnar body 324 can fittingly receive each toe adjusting member 322 made of a rigid material and having different widths to thereby expand the width of each toe columnar body 324. Further, in this embodiment, each of the hallux columnar body 313 and the toe columnar body 324 has a substantially U-shaped cross section, but is not limited thereto. In other embodiments, the hallux columnar body 313 and the toe columnar body 324 may have other shapes.

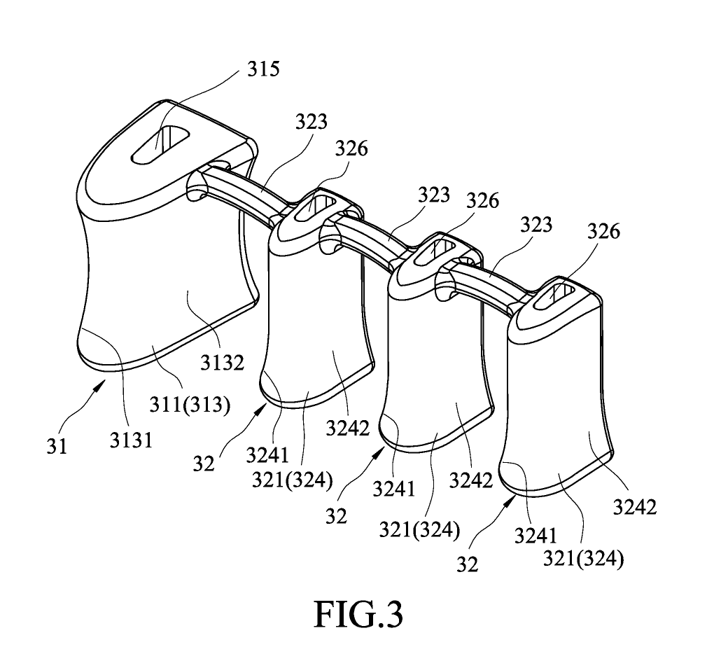

With reference to FIG. 3, the hallux columnar body 313 has a curved surface 3131 for abutting against a web between the hallux and the second toe of the user's foot, and two opposite side surfaces 3132 (only one is visible) extending forwardly from two ends of the curved surface 3131 for abutting against a lateral side of the hallux and a lateral side of the second toe, respectively. Each toe columnar body 324 has a curved surface 3241 for abutting against a web between two adjacent toes excluding the hallux of the user's foot, and two opposite side surfaces 3242 (only one is visible in FIG. 3) extending forwardly from two ends of the curved surface 3241 for abutting against a lateral side of one of the toes and a lateral side of the adjacent toe, respectively.

With reference to FIGS. 2, 4, and 5, the user can choose an appropriate hallux adjusting member 312 according to the extent of his/her toe bone deformities. In the case of a serious overlapping toe deformity, if the web between the hallux and the second toe is forcefully opened, it may cause severe pain and discomfort to the user, so that the hallux adjusting member 312 with a smaller width may first be used and inserted into the first receiving space 314 through the opening 315. Following rehabilitation for a certain period of time, the hallux adjusting member 312 with a smaller width can be replaced with a larger width to further expand the width of the hallux columnar body 313, thereby achieving the effect of separating the toes including the hallux valgus.

Similarly, the user can gradually separate the other toes by choosing the appropriate toe adjusting members 322. Although three toe adjusting members 322 are exemplified in this embodiment, in other variations of this embodiment, the number of the toe adjusting member 322 may be adjusted, and may be one, two or more than four depending on the number of the toes that are affected by the toe bone deformities.

It is worth to mention herein that during selling of this embodiment, it is not limited to providing one hallux adjusting member 312 and three toe adjusting members 322, but a sufficient number of the hallux adjusting member 312 and the toe adjusting member 322 with different widths may be provided to the user to facilitate replacement thereof.

In this embodiment, the upper 4 includes a main body 41 that is in the form of a strap and that has two opposite lateral ends connected to two opposite lateral sides of the sole 2 such that the main body 41 extends across a front portion of the sole 2. The main body 41 cooperates with the sole 2 to define a foot space 100 for receiving the user's foot (not shown). Two apertures 42 are respectively formed in the lateral ends of the main body 41, and are respectively and immediately adjacent to an outer side of the hallux and an outer side of a small toe of the user's foot when the footwear of this disclosure is worn by the user. The toe separating device 3 is located within the foot space 100 with the hallux separating unit 31 and an outermost one of the toe separating units 32 being respectively proximate to the apertures 42.

It should be noted herein that since the toe bone deformities, such as hallux valgus and toe overlapping, are often accompanied by outward protrusion of the outer side of the hallux and the outer side of the small toe, frictions between these parts and an inner side of the upper 4 are increased, so that after a certain period of time, the outer side of the hallux and the outer side of the small toe will develop symptoms, such as broken skin, painfulness, formation of calluses, etc. Thus, through the presence of the apertures 42, the protruding portion of the outer side of the hallux and the protruding portion of the outer side of the small toe can be accommodated in the apertures 42, respectively, and can be prevented from rubbing against the inner side of the upper 4, thereby achieving the effects of relaxing the foot. In addition, in this embodiment, the main body 41 of the upper 4 shields the rear ends of the hallux and the four toes, but exposes the front ends thereof. However, in other embodiments, the main body 41 may be configured to also shield the front ends of the hallux and the four toes.

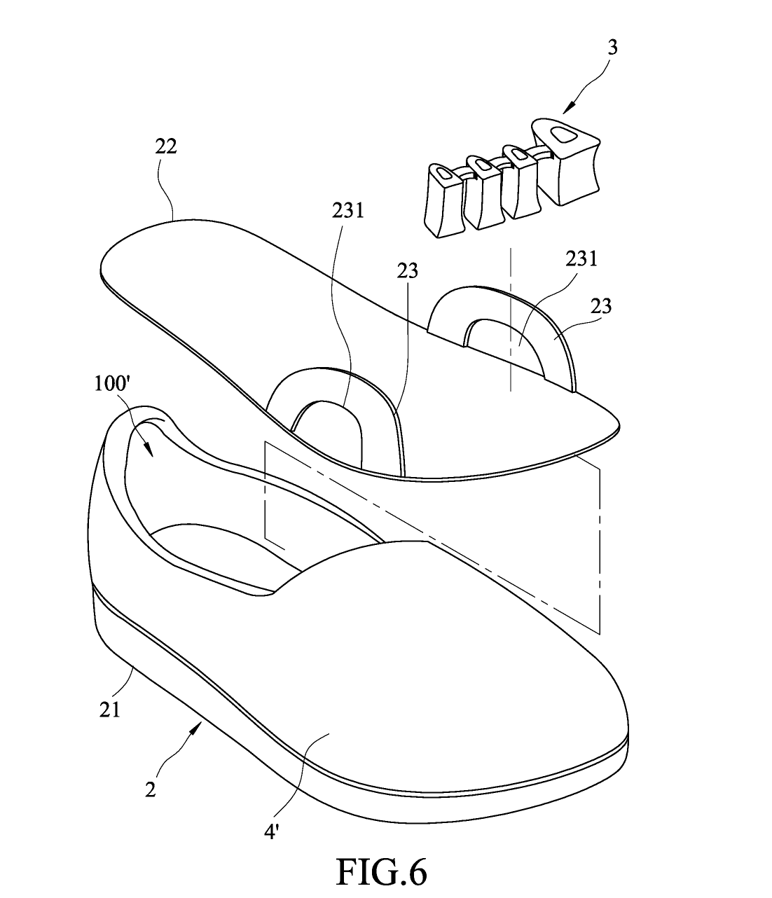

Referring to FIG. 6, a second embodiment of a footwear of this disclosure is shown to be generally identical to the first embodiment. Particularly, the footwear includes a sole 2, a toe separating device 3 and an upper 4′. However, in this embodiment, the footwear is exemplified as a shoe. The sole 2 includes an outsole 21, an insole 22, and two cushion sheets 23 connected respectively and transversely to two opposite lateral sides of the insole 22 and immediately and respectively adjacent to the outer side of the hallux and the outer side of the small toe of the user's foot when the footwear is worn by the user. Each of the cushion sheets 23 has a cutout 231. The upper 4′ has a bottom periphery connected to the outsole 21 so as to cooperate with the outsole 21 to define a foot space 100′ for receiving the user's foot (not shown). The toe separating device 3 is disposed on a top surface of the insole 22, and is located between the cushion sheets 23. The insole 22 together with the toe separating device 3 is inserted into the foot space 100′ such that the insole 22 together with the toe separating device 3 is located on a top surface of the outsole 21 (not shown). In this embodiment, the function of the cutout 231 is similar to the apertures 42 of the first embodiment (see FIG. 2). User of the footwear of this embodiment can similarly achieve the effects of separation of the toes and relaxing of the foot as described in the first embodiment.

In summary, by virtue of using the hallux adjusting member 312 and the toe adjusting member 322 with different widths, the width of the hallux separating column 311 and the width of the toe separating column 321 can be adjusted accordingly so as to separate the toes in a gradual manner and to achieve foot relaxing effect.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A toe separating device, comprising:
a hallux separating unit including a hallux separating column for separating the hallux and the second toe of a user's foot, and a hallux adjusting member, said hallux separating column having a hallux columnar body that is elastically stretchable and that defines a first receiving space having an opening formed in a top surface of said hallux columnar body, said hallux columnar body having a length in a top-bottom direction, and a width in a left-right direction transverse to the top-bottom direction, said hallux adjusting member having a columnar shape and being removably inserted into said first receiving space through said opening to expand the width of said hallux columnar body.

2. The toe separating device as claimed in claim 1, further comprising at least one toe separating unit that includes a toe separating column for separating two adjacent toes excluding the hallux, and a toe adjusting member, said toe separating column having a toe columnar body that is elastically stretchable and that defines a second receiving space having an opening formed in a top surface of said toe columnar body, said toe columnar body having a length in the top-bottom direction, and a width in the left-right direction, said toe adjusting member having a columnar shape and being removably inserted into said second receiving space through said opening of said second receiving space to expand the width of said toe columnar body.

3. The toe separating device as claimed in claim 2, further comprising an arched connecting member interconnecting said toe separating column and said hallux separating column.

4. The toe separating device as claimed in claim 2, wherein said toe separating device comprises three said toe separating units, and further comprises three arched connecting members, one of said arched connecting members interconnecting said toe separating column of one of said toe separating units and said hallux separating column, each of the other two of said arched connecting members interconnecting said toe separating columns of two adjacent ones of said toe separating units, said hallux separating column and said toe separating columns being arranged spaced apart from each other along the left-right direction.

5. The toe separating device as claimed in claim 1, wherein said hallux columnar body has a substantially U-shaped cross section, and has a curved surface for abutting against a web between the hallux and the second toe of the user's foot, and two opposite side surfaces extending forwardly from two ends of said curved surface for abutting against a lateral side of the hallux and a lateral side of the second toe of the user's foot, respectively.

6. A footwear, comprising:
a sole; and
a toe separating device disposed on a top surface of said sole and including
a hallux separating unit including a hallux separating column for separating the hallux and the second toe of a user's foot, and a hallux adjusting member, said hallux separating column having a hallux columnar body that is elastically stretchable and that defines a first receiving space having an opening formed in a top surface of said hallux columnar body, said hallux columnar body having a length in a top-bottom direction, and a width in a left-right direction transverse to the top-bottom direction, said hallux adjusting member having a columnar shape and being removably inserted into said first receiving space through said opening to expand the width of said hallux columnar body.

7. The footwear as claimed in claim 6, wherein said toe separating device further comprises at least one toe separating unit that includes a toe separating column for separating two adjacent toes excluding the hallux, and a toe adjusting member, said toe separating column having a toe columnar body that is elastically stretchable and that defines a second receiving space having an opening formed in a top surface of said toe columnar body, said toe columnar body having a length in the top-bottom direction, and a width in the left-right direction, said toe adjusting member having a columnar shape and being removably inserted into said second receiving space through said opening of said second receiving space to expand the width of said toe columnar body.

8. The footwear as claimed in claim 7, wherein said toe separating device further includes an arched connecting member interconnecting said toe separating column and said hallux separating column.

9. The footwear as claimed in claim 7, wherein said toe separating device includes three said toe separating units, and further includes three arched connecting members, one of said arched connecting members interconnecting said toe separating column of one of said toe separating units and said hallux separating column, each of the other two of said arched connecting members interconnecting said toe separating columns of two adjacent ones of said toe separating units, said hallux separating column and said toe separating columns being arranged spaced apart from each other along the left-right direction.

10. The footwear as claimed in claim 6, further comprising an upper that includes a main body connected to said sole and cooperating with said sole to define a foot space for receiving the user's foot.

11. The footwear as claimed in claim 10, wherein said main body has two opposite lateral ends connected to two opposite lateral sides of said sole, and has two apertures respectively formed in said lateral ends and respectively and immediately adjacent to an outer side of the hallux and an outer side of a small toe of the user's foot.

12. The footwear as claimed in claim 10, wherein said sole includes an outsole, an insole, and two cushion sheets connected respectively and transversely to two opposite lateral sides of said insole and immediately and respectively adjacent to an outer side of the hallux and an outer side of a small toe of the user's foot, each of said cushion sheets having a cutout, said upper having a bottom periphery connected to said outsole so as to cooperate with said outsole to define said foot space, said toe separating device being disposed on a top surface of said insole, said insole together with said toe separating device being inserted into said foot space such that said insole together with said toe separating device is located on a top surface of said outsole.

13. The footwear as claimed in claim 6, wherein said hallux columnar body has a substantially U-shaped cross section, and has a curved surface for abutting against a web between the hallux and the second toe of the user's foot, and two opposite side surfaces extending forwardly from two ends of said curved surface for abutting against a lateral side of the hallux and a lateral side of the second toe of the user's foot, respectively.
